(19) United States
(12) Patent Application Publication
(10) Pub. No.: US 2019/0201350 A1
(43) Pub. Date: Jul. 4, 2019

(54) TIME RELEASE OF FAT-SOLUBLE ACTIVES

(71) Applicant: Corr-Jensen Inc., New York, NY (US)

(72) Inventors: Tyler O. White, Greenwood, SC (US); Matthew Hesse, New York, NY (US)

(21) Appl. No.: 16/325,670

(22) PCT Filed: Aug. 14, 2017
(86) PCT No.: PCT/US2017/046733

§ 371 Date: Feb. 14, 2019

Related U.S. Application Data
(60) Provisional application No. 62/375,147, filed on Aug. 15, 2016.

(51) Int. Cl. A61K 9/50; A61K 31/065; A61K 31/353

(52) U.S. Cl. CPC A61K 9/5015; A61K 9/501; A61K 9/5036; A61K 31/353; A61K 9/5052; A61K 9/5063; A61K 31/065; A61K 9/5042

(57) ABSTRACT

Compositions include an extended release fat-soluble active composition. In certain aspects, the extended release fat-soluble active composition includes an extended release bead multiparticulate comprising a fat-soluble active. In other aspects, the extended release fat-soluble active composition includes an extended release lipid multiparticulate comprising a fat-soluble active.

### CROSS REFERENCE TO RELATED APPLICATIONS

This application depends from and claims priority to U.S. Provisional Application No. 62/375,147 filed Aug. 15, 2016, the entire contents of which are incorporated herein by reference.

### FIELD

The invention relates to time-release compositions of fat-soluble actives, as well as methods for their preparation and use.

### BACKGROUND

Fat-soluble actives, such as fat-soluble vitamins, carotenoids, and polyunsaturated fatty acids can enhance nutrition and provide many provide many additional benefits. For example, such fat soluble actives can provide a protective effect or prevent various cancers, provide an analgesic effect, provide an anti-psychotic effect, relieve convulsions, relieve inflammation, relieve anxiety, relieve nausea, treat symptoms of multiple sclerosis, reduce the risk of a heart attack, reduce the risk of cardiovascular diseases, and decrease the risk of developing amyotrophic lateral sclerosis

Although there are various techniques to provide formulations of fat-soluble actives as orally administrable forms, there is a need in the art to provide a sustained and controlled supply of fat-soluble actives to individuals.

### SUMMARY

It is understood that both the following summary and the detailed description are exemplary and explanatory and are intended to provide further explanation of the disclosure as claimed. Neither the summary nor the description that follows is intended to define or limit the scope of the disclosure to the particular features mentioned in the summary or description.

One object is to provide a sustained and controlled supply of fat-soluble actives to individuals. This object is achieved in the present disclosure that provides an extended release fat soluble active composition. In certain aspects, the extended release fat-soluble active composition includes an extended release bead multiparticulate comprising a fat-soluble active. In other aspects, the extended release fat-soluble active composition includes an extended release lipid multiparticulate comprising a fat soluble active and lipid matrix.

Another aspect provides a process for enhancing and sustaining a supply of a fat-soluble in a subject. The process comprises administering to the subject an extended release fat-soluble active composition and enhancing and sustaining a supply of a fat-soluble active in said subject by said step of administering. In certain aspects, the extended release fat-soluble composition includes an extended release bead multiparticulate comprising a fat-soluble active.

In other aspects, the extended release fat-soluble composition includes an extended release lipid multiparticulate comprising a fat-soluble active.

### DETAILED DESCRIPTION

The following description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the compositions or processes are described as using specific materials or an order of individual steps, it is appreciated that materials or steps may be interchangeable such that the description of the invention may include multiple parts or steps arranged in many ways as is readily appreciated by one of skill in the art.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for describing particular aspect only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Provided are compositions that include an extended release fat-soluble active composition. In certain aspect, the extended release fat-soluble active composition includes an extended release bead multiparticulate comprising a fat-soluble active. In other aspects, the extended release fat-soluble active composition includes an extended release lipid multiparticulate comprising a fat-soluble active. Such compositions can be used for enhancing and sustaining a supply of a fat-soluble active in a subject. Additionally, depending on the fat-soluble active(s) in the composition, such compositions can be used for enhancing nutrition, providing a protective effect or prevent various cancers, providing an analgesic effect, providing an anti-psychotic effect, relieving convulsions, relieving inflammation, relieving anxiety, relieve nausea, treating symptoms of multiple sclerosis, reducing the risk of a heart attack, reducing the risk of cardiovascular diseases, and decreasing the risk of developing amyotrophic lateral sclerosis.

As such, a composition includes an extended release component comprising a fat-soluble active. The extended release component comprises a fat-soluble active formulated for sustained release, such that the composition provides a sustained and enhanced supply of a fat-soluble active. In certain aspects, the extended release component comprises a fat-soluble active formulated for sustained release, delayed release, or both, such that the extended release component provides a sustained supply of a fat-soluble active, a latter burst of a fat-soluble active, or combinations thereof. In some aspects, the extended release component comprises an extended release bead multiparticulate, the extended release bead multiparticulate comprising a fat-soluble active. In other aspects, the extended release fat-soluble composition includes an extended release lipid multiparticulate, the extended release lipid multiparticulate comprising a fat-soluble active. In some aspects, the extended release fat-soluble active composition is an oral dosage form. In particular aspects, the extended release fat-soluble active composition is a two-piece liquid capsule.

In some aspects, an extended release fat-soluble active composition includes an extended release component comprising a fat-soluble active. In certain aspects, the extended release composition comprises an extended release component comprising a fat-soluble active formulated for sustained release, delayed release, or both, such that the extended release component provides a sustained supply of a fat-soluble active, a latter burst of a fat-soluble active, or combinations thereof. In some aspects, the extended release component comprises extended release beads, delayed release beads, or beads providing both extended and delayed release. In other aspects, the extended release component comprises an extended release lipid multiparticulate, the extended release lipid multiparticulate comprising a fat-soluble active and a lipid matrix.

A fat-soluble active is a physiologically active ingredient that is soluble in lipids and insoluble or sparingly soluble in water. In some aspects, the fat-soluble active of the extended release component can comprise fat-soluble vitamins and fat-soluble vitamin derivatives; carotenoids and carotenoid derivatives; cannabinoids; polyunsaturated fatty acids e.g. eicosapentaenoic acid, docosahexaenoic acid, and their triglyceride esters; or various mixtures or combinations thereof.

In certain aspects, fat-soluble vitamins include, but are not limited to, vitamin A, vitamin D, vitamin E, vitamin K, vitamin F, and derivatives thereof such as vitamin A esters, e.g. vitamin A acetate and palmitate, and vitamin E esters, e.g. tocopherol acetate. An extended release fat-soluble active composition that includes a fat-soluble vitamin can be utilized to enhance nutrition in a subject. Fat-soluble vitamins are commercially available from sources known by those of skill in the art.

In certain aspects, carotenoids and carotinoid derivatives include, but are not limited to, α- or β-carotene, astaxantin, lutein, zeaxanthin, cryptoxanthin, 8′-apo-β-carotenal, 8′-apo-β-carotenoic acid esters such as the ethyl ester, canthaxanthin, astaxanthin, astaxanthin esters, lycopene, lutein, zeaxanthin or crocetin and their derivatives. An extended release fat-soluble active composition that includes a carotenoid can be utilized to provide a protective effect or prevent cancer in a subject, including lung cancer and prostate cancer. Carotenoids and carotinoid derivatives are commercially available from sources known by those of skill in the art.

Cannabinoids are a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids. Phytocannabinoids are compounds produced by the Cannabis plant. Cannabinoids are divided into different classes based on small differences in the ring structures; cannabinols, cannabidiols, and tetrahydrocannabinols. Different cannabinoid species in the same class are distinguished by various lengths of the C-3 side chain attached to the aromatic ring. Photocannabinoids are are known to be agonists for cannabinoid receptors CB1 and CB2.

The cannabinoid class of cannabinols is represented by Formula I, wherein R at the C-3 position is selected from C₁-C₃₀ alkyl, C₂-C₃₀ alkenyl or C₂-C₃₀ alkynyl, optionally substituted with substituents. In certain aspects, R is C₁-C₃₀ alkyl or C₂-C₃₀ alkenyl. In other aspects, R is C₁-C₅ alkyl or alkenyl, such as C₁, C₂, C₃, C₄ or C₅ alkyl or alkenyl. In certain aspect, alkenyl is a group having one, two or three double bonds. An extended release fat-soluble active composition that includes a cannabinol can be utilized to provide an analgesic effect in a subject. Cannabinols can be obtained commercially, e.g., from Avanti Polar Lipids, Tocris Bioscience, Biomol, Cayman Chemicals, Sigma-Aldrich or Fluka.

The generic structural formula for cannabinols is as follows:

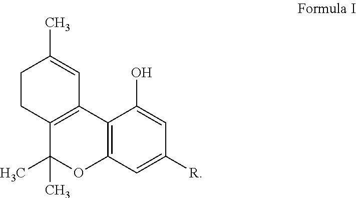

Cannabidiols include one of at least 85 active cannabinoids identified in cannabis.

Cannabidiol is a major phytocannabinoid, accounting for up to 40% of the plant's extract. Cannabidiols are considered to have a wider scope of medical applications than tetrahydrocannabinols (THC). Cannabidiols have a very low affinity for CB1 and CB2 receptors but acts as an indirect antagonist of their agonists. Cannabidiols may potentiate THC's effects by increasing CB1 receptor density or through another CB1-related mechanism. Cannabidiols are also an inverse agonist of CB2 receptors. Cannabidiols possess antiproliferative, pro-apoptotic effects and inhibit cancer cell migration, adhesion and invasion. Cannabidiols are well known in the art, and are represented by Formula II depicted below, wherein R is selected from Ci-C₃₀ alkyl, C₂-C₃₀ alkenyl or C₂-C₃₀ alkynyl, optionally substituted with substituents. In some aspects, R is C₁-C₃₀ alkyl or C₂-C₃₀ alkenyl. In other aspects, R is C₁-C₅ alkyl or alkenyl, such as C₁, C₂, C₃, C₄ or C₅ alkyl or alkenyl. In certain aspects, R is a C₅ alkyl. In some aspects, an alkenyl is a group having one, two or three double bonds. An extended release fat-soluble active composition that includes a cannabidiol can be utilized to provide an anti-psychotic effect in a subject, as well as relieve convulsions, inflammation, anxiety, and nausea in a subject. Furthermore, an extended release fat-soluble active composition that includes a cannabinol can be utilized to treat various tumors, including but not limited to, neuroblastoma, mantle cell lymphoma, colon cancer, osteosarcoma, and glioma. Cannabidiols can be obtained commercially, e.g., from Avanti Polar Lipids, Tocris Bioscience, Biomol, Cayman Chemicals, Sigma-Aldrich or Fluka.

The generic structural formula for cannabidiols is as follows:

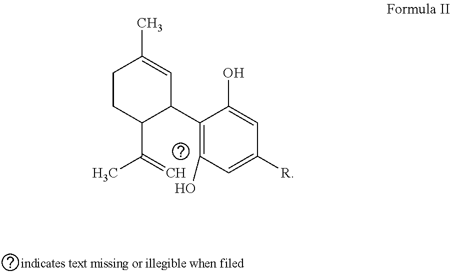

The cannabinoid class of tetrahydrocannabinols have the general structure depicted in Formula III, wherein R is selected from C₁-C₃₀ alkyl, C₂-C₃₀ alkenyl or C₂-C₃₀ alkynyl, optionally substituted with substituents. In some aspects, R is C₁-C₃₀ alkyl or C₂-C₃₀ alkenyl. In other aspects, R is C₁-C₅ alkyl or alkenyl, such as C₁, C₂, C₃, C₄ or C₅ alkyl or alkenyl. In certain aspects, R is a C₅ alkyl. The double bond in the first ring can be either in the Δ⁸ (as depicted) or Δ⁹ position. An extended release fat-soluble active composition that includes a tetrahydrocannabinol can be utilized to: provide an analgesic effect, as a sleep aid, an appetite stimulant, to treat glaucoma, to relieve anxiety, and relieve nausea in a subject. An extended release fat-soluble active composition that includes a tetrahydrocannabinol can also be utilized to treat symptoms of multiple sclerosis, including treating central pain and painful spasms and for improvement of objective measures of spasticity. Tetrahydrocannabinols can be prepared, e.g. by extraction from a cannabis plant. Also, tetrahydrocannabinols can be obtained commercially, e.g., from Tocris Bioscience, Biomol, Cayman Chemicals, Sigma-Aldrich or Fluka.

The generic structural formula of tetrahydrocannabinols is as follows:

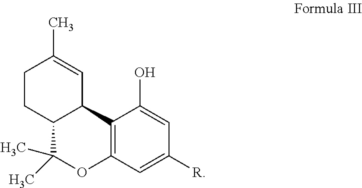

In certain aspects, polyunsaturated fatty acids include, but are not limited to omega-3 polyunsaturated fatty acids (e.g., eicosapentaenoic acid, docosahexaenoic acid, and their triglyceride esters) omega-6 polyunsaturated fatty acids, and omega 9 polyunsaturated fatty acids. An extended release fat-soluble active composition that includes a polyunsaturated fatty acid can be utilized to provide a protective effect or prevent cancer in a subject, including lung breast cancer, as well as lower the risk of a heart attack, reduce the risk of cardiovascular diseases, and decrease the risk of developing amyotrophic lateral sclerosis. Polyunsaturated fatty acids are commercially available from sources known by those of skill in the art.

The term “extended release” refers to the gradual release of the fat-soluble active (including fat-soluble vitamins and fat-soluble vitamin derivatives; carotenoids and carotinoid derivatives; cannabinoids; polyunsaturated fatty acids e.g., eicosapentaenoic acid, docosahexaenoic acid, and their triglyceride esters; or various mixtures or combinations thereof) from the extended release component (including extended release beads and/or extended release lipid multiparticulate) of the composition over an extended period of time, optionally greater than 30 minutes where extended release is measured in a simulated fed state medium including 4 wt % caprylocaproyl polyoxyl-8 glycerides (Labrasol®) and 2 wt % macrogolglycerol ricinoleate (Kolliphor® EL) in water. With extended release, the rate of release of the fat-soluble active from the extended release component (including extended release beads and/or extended release lipid multiparticulate) is reduced in order to maintain therapeutic activity of the fat-soluble active for a longer period of time. As described herein, an “extended release” component preferably releases not less than 80% of the fat-soluble active in about 12 hours, e.g., in about 12 hours, in about 11 hours, in about 10 hours, in about 9 hours, in about 8 hours, in about 6 hours, in about 4 hours, or any value or range therebetween. In certain aspects, the “extended release” component preferably releases about 100% of the fat-soluble active in about 24 hours, e.g. in about 24 hours, in about 22 hours, in about 20 hours, in about 18 hours, in about 16 hours, in about 14 hours, in about 12 hours, in about 11 hours, in about 10 hours, in about 9 hours, in about 8 hours, in about 6 hours, in about 4 hours, or any value or range therebetween. In certain aspects, an “extended release” component preferably releases not more than 20% of the fat-soluble active in about 1 hour, in about 50 minutes, in about 40 minutes, in about 30 minutes, in about 20 minutes, or any value or range therebetween. In other aspects, an “extended release” component preferably releases not more than 10% of the fat-soluble active in about 1 hour, in about 50 minutes, in about 40 minutes, in about 30 minutes, in about 20 minutes, or any value or range therebetween.

In certain aspects, the extended release composition can comprise delayed release component, such as delayed release beads or delayed release lipid multiparticulates. The term “delayed release” refers to modified release in which the release of the fat-soluble active (including fat-soluble vitamins and fat-soluble vitamin derivatives; carotenoids and carotinoid derivatives; cannabinoids; polyunsaturated fatty acids e.g. eicosapentaenoic acid, docosahexaenoic acid, and their triglyceride esters; or various mixtures or combinations thereof) from the delayed release component of the composition is delayed after oral administration for a finite period of time after which release of the drug is unhindered.

In certain aspects, the extended release beads, including extended release beads, delayed release beads, or beads providing both extended and delayed release, comprise an inert core and a nutrient layer coating the inert core. The inert core of the extended release beads can comprise at least one of celluloses, starches, saccharides, or mixtures thereof. In certain aspects, the inert core of the extended release beads is spherical. In other aspects, the inert core of the extended release beads is a sugar sphere, as are known in the art and commercially available.

In some aspects, the nutrient layer comprises a fat-soluble active (including fat-soluble vitamins and fat-soluble vitamin derivatives; carotenoids and carotinoid derivatives; cannabinoids; polyunsaturated fatty acids e.g. eicosapentaenoic acid, docosahexaenoic acid, and their triglyceride esters; or various mixtures or combinations thereof).

In certain aspects, the nutrient layer can comprise one or more water soluble vitamins and minerals in addition to a fat-soluble active. The one or more water soluble vitamins and minerals can comprise at least one of Vitamin B1 (optionally in the form of Thiaine Mononitrate), Vitamin B2 (optionally in the form of Riboflavin), Vitamin B3 (optionally in the form of Niacin), Vitamin B5 (optionally in the form of Pantothenic Acid), Vitamin B6 (optionally in the form of Pyridoxine HCL), Folic Acid, Vitamin B12 (optionally in the form of Methylcobalamin), Vitamin C (optionally in the form of Ascorbic Acid), Biotin, Calcium, Iron (optionally in the form of Ferrous Sulfate Monohydate), Phosphorus, Sulfer, Zinc (optionally in the form of Zinc Oxide), Copper (optionally in the form o Cupric Oxide), Iodine (Optionally in the form of Potassium Iodine), Manganese (optionally in the form of manganese gluconate), Chromium (optionally in the form of Chromium Chelate), Molybdenum (optionally in the form of Molybdenum Chelate), Selenium (optionally in the form of Sodium Selenate), Choline, Fluoride, Chloride, Potassium, Sodium, and various mixtures or other combinations thereof. Water soluble vitamins and/or minerals are commercially available from sources known by those of skill in the art.

In even further aspects, the nutrient layer can comprise a “performance enhancing component” in addition to a fat-soluble active. A performance enhancing component is intended to encompass one or more of a: vitamin (e.g. vitamin B12 (optionally in the form of methycobalamin available from Anmar), vitamin B3 (optionally in the form of nicinaminde/nicotinamide available from DSM) among others); beta-alanine or derivative thereof (optionally CARNOSYN available from Natural Alternatives Intl.), mineral; protein; amino acid (e.g. arginine (optionally in the form of arginine silicate inositol available as Nitrosigine from Nutrition 21, or agmatine sulfate available from Parchem) glutamine (available from Kyowa Hakko), creatine (optionally in the form of creatine HCL available from Pharmline), carnitine, glycine, trimethyl glycine, tyrosine, leucine (available from Glanbia or Danisco), isoleucine (available from Glanbia), valine (available from Glanbia), citrulline (optionally in the form of citrulline malate available from Creative Compounds), among others or derivatives thereof optionally N-acetyl L-tyrosine (available from Cepham); branched-chain amino acids (optionally in the form of Pepform 2:1:1 BCAA containing a 2:1:1 ratio of leucine, isoleucine and valine, available from Glanbia); astragalus membranaceus and panax notoginseng carbohydrate (optionally in the form of Astragin available from N Liv Science); fatty acid (optionally essential fatty acid); stimulant illustratively caffeine (optionally 1,3,7-trimethylxanthine available from Mitsubishi), ephedrine, or forskholin; pyruvate; a citric acid cycle intermediate; betaine (optionally in the form of betaine anhydrous available from Danisco), norvaline (optionally in the form of L-novaline, available from Cepham), one or more plant components such as an essential oil or plant extract (illustratively citrus aurantium, grape seed extract, or piper nigrum (available from Indena)), black pepper extract (optionally BioPerine® or more specifically Black Pepper PE 95% BioPerine® from Sabinsa Corporation), ashwagandha extract (optionally KSM66 from Ixoreal)), or a derivative of any of the foregoing.

In one aspect, the nutrient layer can be applied, e.g., as an aqueous suspension or dispersion, over the inert core, a binding layer, or a seal layer of the extended release beads, including extended release beads, delayed release beads, or beads providing both extended and delayed release, and forms a separate layer thereon

In certain aspects, the extended release beads, including extended release beads, delayed release beads, or beads providing both extended and delayed release, further comprise a seal film layer. In further aspects, the seal film layer comprises a sealer. Suitable sealers include, but are not limited to, celluloses such as hydroxypropylmethylcellulose, hydroxypropyl cellulose, microcrystalline cellulose and carboxymethyl-cellulose sodium, polyvinyl pyrrolidone, polyethylene glycol, starches, and combinations thereof. In certain aspects, the seal film layer coats the nutrient layer. In further aspects, the seal film layer directly coats the nutrition layer. In one aspect, the seal film layer can be applied, e.g., as an aqueous suspension or dispersion, over the nutrient layer, a binder layer, or inert core of the extended release beads, including extended release beads, delayed release beads, or beads providing both extended and delayed release, and forms a separate layer thereon.

In certain aspects, the extended release beads, including extended release beads, delayed release beads, or beads providing both extended and delayed release, further comprise at least one binder. Binders are substances that are useful in holding other excipients or active ingredients together as solids. Suitable binders include, but are not limited to, celluloses such as hydroxypropylmethylcellulose, hydroxypropyl cellulose, microcrystalline cellulose and carboxymethyl-cellulose sodium, polyvinyl pyrrolidone, polyethylene glycol, starches, and combinations thereof. In certain aspects, the at least one binder is included in the nutrient layer. In other aspects, the at least one binder can be included in a binder layer of the extended release beads. In one aspect, the binder layer can be applied, e.g., as an aqueous suspension or dispersion, over the nutrient layer, seal film layer, or inert core of the extended release beads, including extended release beads, delayed release beads, or beads providing both extended and delayed release, and forms a separate layer thereon.

The extended release beads, delayed release beads, or both extended and delayed release beads of the composition can be manufactured using methods that are known in the art. Such methods include, but are not limited to, dry and wet granulation technology, including fluid bed granulation, high shear granulation, extrusion and spheronization, coating an inert core (including but not limited to, fluid bed coating an inert core), and spay drying layers over an inert core, as are known in the art. Methods for forming beads are described in U.S. Pat. No. 6,066,334 and U.S. Pat. No. 6,046,277, U.S. Pat. No. 6,046,277, U.S. Pat. No. 6,001,392, US2007/0215511, US2005/232986, US2005/232987 US2005/232993, US2005/266032, and US2003/009971.

In some aspects, the extended release lipid multiparticulates comprise a fat-soluble active and a lipid matrix. In certain aspects, the fat-soluble active is incorporated into the lipid matrix. A fat-soluble active may be fully solubilized, partially solubilized, or suspended in the lipid matrix. In certain aspects, the lipid matrix comprises natural and/or synthetic oils, fatty acids and their derivatives, glycerides, fatty acid esters, glycolized fatty acid esters, fatty alcohols, sterols, waxes, hard fat, and/or combination thereof.

Exemplary natural oils include, but are not limited to, vegetable oil such as sunflower oil, olive oil, groundnut oil, and palm oil, as well as hydrogenated vegetable oils, including hydrogenated cottonseed oil. Exemplary synthetic oils include, but are not limited to, hydrophobic silicone, cyclomethicones, petroleum waxes or jellies, linear alkanes, lipophilic organic fluorinated oils, perhydrosqualene and/or mixtures thereof.

Exemplary fatty acids include, but are not limited to, stearic acid, benzoic acid, citric acid, fumaric acid, lactic acid, and maleic acid. Exemplary glycerides include, but are not limited to, monoglycerides, diglycerides, triglycerides, and combinations thereof, etc. with saturated or unsaturated chains having carbon numbers from C6 to C40, e.g. C18 to C24, C8 to C32, C10 to C24, C10 to C18, C12 to C18, etc.), hemisynthetic glycerides or glyceride derivatives with saturated or unsaturated medium to long chain lengths. Exemplary long-chain fatty acid esters include, but are not limited to, glyceryl monooleate, glyceryl monostearate, glycerol behenate, glycerol monostearate, glyceryl palmitostearate, mixtures of mono-, di-, and trialkyl glycerides, including mixtures of glyceryl mono-, di-, and tribehenate (e.g. available commercially as Compritol® products from Gattefosse), glyceryl tristearate, and glyceryl tripalmitate. Exemplary non-neutralized fatty acid include, but are not limited to, fatty acids with linear chains with carbon numbers ranging from C4 to C18, for example such as myristic acid, lauric acid, palmitic acid, or oleic acid and mixtures thereof

Exemplary short to medium chain fatty acid esters include, but are not limited to, isopropyl palmitate, isopropyl myristate, triethyl citrate, lecithin, triacetin, and dibutyl sebacate. Esters of fatty acids with carbon numbers from C6 to C12 with glycols, e.g. ethylene glycol, propylene glycol, may also be used. Glycolized fatty acid esters include, but are not limited to, polyethylene glycol stearate and polyethylene glycol distearate.

Exemplary sterols include, but are not limited to, cholesterol and its esters. Exemplary waxes include, but are not limited to Carnauba wax, Candellila wax, Alfa wax, vegetable waxes, rice wax, hydrogenated jojoba wax or florali absolute waxes, beeswaxes and modified beeswaxes, microcrystalline wax, and paraffin wax. Exemplary fatty alcohols include fatty alcohols with high molecular weight (e.g. cetanol, myristoyl alcohol, stearyl alcohol).

Esters of acids and alcohols with high molecular weight include, but are not limited to, esters of linear and saturated acids with even carbon numbers from C14 to C20, and linear and saturated alcohols with even carbon numbers from C14 to C32.

The matrix material may comprise mixtures of materials, such as mixtures of any of the foregoing.

In certain aspects, lipid matrix materials include an alkyl-containing glycerol such as a mixture of mono-, di- and triglyceryl behenates (commercially available as COMPRITOL 888, Suppocire ®, a semi-synthetic glyceride base comprising saturated C₈-C₁₈ triglyceride fatty acids and Precirol ® (glyceryl distearate) from Gattefose Corporation of Westwood, N.J.); and hydrogenated cottonseed oil (commercially available as LUBRITAB from Edward Mendell Co. of Patterson, N.Y.).

The lipid matrix may also include clays or their oily dispersions, gums of phenylated silicones, starches, and/or fat structuring agents for the purpose of adjusting consistency. The lipid matrix may also include a certain number of compounds such as mineral fillers, to modulate density and plasticity. The mineral fillers may be, for example, talc and/or kaolin.

The amount of lipid matrix present in the solid lipid particles may be at a weight percent of the total weight of the solid lipid particles of from 1% to 90%, from 1% to 75%, 25% to 70%, or any value or range in between.

In further aspects, the extended release lipid multiparticulates can comprise one or more water soluble vitamins and minerals in addition to a fat-soluble active. The one or more water soluble vitamins and minerals can comprise at least one of Vitamin B1 (optionally in the form of Thiaine Mononitrate), Vitamin B2 (optionally in the form of Riboflavin), Vitamin B3 (optionally in the form of Niacin), Vitamin B5 (optionally in the form of Pantothenic Acid), Vitamin B6 (optionally in the form of Pyridoxine HCL), Folic Acid, Vitamin B12 (optionally in the form of Methylcobalamin), Vitamin C (optionally in the form of Ascorbic Acid), Biotin, Calcium, Iron (optionally in the form of Ferrous Sulfate Monohydate), Phosphorus, Sulfer, Zinc (optionally in the form of Zinc Oxide), Copper (optionally in the form o Cupric Oxide), Iodine (Optionally in the form of Potassium Iodine), Manganese (optionally in the form of manganese gluconate), Chromium (optionally in the form of Chromium Chelate), Molybdenum (optionally in the form of Molybdenum Chelate), Selenium (optionally in the form of Sodium Selenate), Choline, Fluoride, Chloride, Potassium, Sodium, and various mixtures or other combinations thereof. Water soluble vitamins and/or minerals are commercially available from sources known by those of skill in the art.

In even further aspects, the extended release lipid multiparticulates can comprise a performance enhancing component in addition to a fat-soluble active. A performance enhancing component is intended to encompass one or more of a: vitamin (e.g. vitamen B12 (optionally in the form of methycobalamin available from Anmar), vitamin B3 (optionally in the form of nicinaminde/nicotinamide available from DSM) among others); beta-alanine or derivative thereof (optionally CARNOSYN available from Natural Alternatives Intl.), mineral; protein; amino acid (e.g. arginine (optionally in the form of arginine silicate inositol available as

Nitrosigine from Nutrition 21, or agmatine sulfate available from Parchem) glutamine (available from Kyowa Hakko), creatine (optionally in the form of creatine HCL available from Pharmline), carnitine, glycine, trimethyl glycine, tyrosine, leucine (available from Glanbia or Danisco), isoleucine (available from Glanbia), valine (available from Glanbia), citrulline (optionally in the form of citrulline malate available from Creative Compounds), among others or derivatives thereof optionally N-acetyl L-tyrosine (available from Cepham); branched-chain amino acids (optionally in the form of Pepform 2:1:1 BCAA containing a 2:1:1 ratio of leucine, isoleucine and valine, available from Glanbia); astragalus membranaceus and panax notoginseng carbohydrate (optionally in the form of Astragin available from N Liv Science); fatty acid (optionally essential fatty acid); stimulant illustratively caffeine (optionally 1,3,7-trimethylxanthine available from Mitsubishi), ephedrine, or forskholin; pyruvate; a citric acid cycle intermediate; betaine (optionally in the form of betaine anhydrous available from Danisco), norvaline (optionally in the form of L-novaline, available from Cepham), one or more plant components such as an essential oil or plant extract (illustratively citrus aurantium, grape seed extract, or piper nigrum (available from Indena)), black pepper extract (optionally BioPerine® or more specifically Black Pepper PE 95% BioPerine® from Sabinsa Corporation), ashwagandha extract (optionally KSM66 from Ixoreal)), or a derivative of any of the foregoing.

The one or more vitamins and minerals and performance enhancing component of the extended release lipid multiparticulates can incorporated into the lipid matrix, and may be fully solubilized, partially solubilized, or suspended in the lipid matrix.

The extended release lipid multiparticulates the composition can be manufactured using methods that are known in the art, e.g., extrusion/spheronization and a melt-spray congeal process. For example, solid lipid particles can be obtained by mixing a lipid phase under moderate heat. For example, wax and oil may be mixed at a temperature corresponding to the melting point of the wax, until the mixture obtained has a melting point lower than the melting point of the wax. The initial ratio of the wax to the oil can be modulated so that the melting point of the end mixture is lower than the degradation temperature of the most heat-sensitive component to be incorporated. In one aspect, the end mixture is a solid at the temperature of its utilization. For example, the end mixture may have a melting point of 37.5° C. The end mixture is cooled while stirring to a temperature slightly above its melting point, e.g. 2° C. or 3° C. above its melting point. One or more active ingredients may then be added. In other aspects, addition of the active ingredient to the mixture is accomplished such that the ingredient is dispersed throughout—such means include the use of a homogenizer, disperser, or the use of mechanical agitation or stirring. Sonicators or static mixers may be also be used. Other ingredients may be similarly incorporated at the same or different times. The mixture is then shaped to give solid lipid particles. Shaping can be carried out using a gel, as is known in the art. Examples of gelifying agents include carboxyvinyl polymers such as polyacrylic polymers not modified by hydrophobic groups or surfactant groups. Other gelifying agents include carrageenans, thickeners and polysaccharidic gels such as xanthenes, guar and carob gels, alginates, cellulose derivatives, pectins, agar, etc. or mixtures thereof. The gel may be prepared with a gel-forming/gelifying, shear-thinning, non-surface-active agent or substance, with which the mixture is not miscible. The mixture may be injected into the gel, for example, through an orifice located at the base of a reaction vessel holding the gel, to form a dispersion. Stirring may be continued throughout injection using a blade equipped with an anchor designed to disperse the mixture and a second axial blade equipped with a three-vaned impeller designed to obtain a dispersion having a desired droplet size or a dispersion having a discontinuous phase of a desired characteristic size. The temperature of the gel may be adjusted to be the same as the temperature of the mixture prior to injection. The dispersion may then be cooled below the melting point of the mixture. Solid lipid particles may then be separated from the gel, after which, the solid lipid particles may be washed. Methods for forming lipid multiparticulates are described in WO 1999/65448, WO 2004/084856, U.S. 6,572,892; WO 2006070117, U.S. Pat. No. 7,625,507, U.S. Pat. No. 7,951,403, U.S. Pat. No. 7,736,672, and U.S. Pat. No. 7,887,844.

Depending on the fat-soluble active(s) in the composition, the fat-soluble active and optionally the one or more vitamins and minerals and performance enhancing component of the extended release component, such as the extended release beads (including extended release beads, delayed release beads, or beads providing both extended and delayed release) and/or the extended release lipid multiparticulate, is present to provide an in vivo concentration effective to enhance nutrition, provide a protective effect or prevent various cancers, provide an analgesic effect, provide an anti-psychotic effect, relieve convulsions, relieve inflammation, relieve anxiety, relieve nausea, treat symptoms of multiple sclerosis, reduce the risk of a heart attack, reduce the risk of cardiovascular diseases, and decrease the risk of developing amyotrophic lateral sclerosis. In other aspects, a fat-soluble active, and optionally the one or more vitamins and minerals and performance enhancing component, of the extended release beads (including extended release beads, delayed release beads, or beads providing both extended and delayed release) and/or extended release lipid multiparticulate, is present to provide an in vivo concentration effective to enhance nutrition and/or to improve athletic performance. As used herein, the term “performance” means performance in athletics. Performance means strong, precise, controlled movements over the time desired by an athlete to achieve a particular result of strength, speed, power and/or precision. “Athlete” is herein defined as a mammal who performs such movements, either in competition, for recreation, or in studies. Athletes illustratively include but are not limited to cyclists, swimmers, bodybuilders, racehorses, racing dogs, and the like. An increase in athletic performance is measured as higher power output, more stamina, or faster speed, optionally in combination with precision of movement or an increase in frequency of performance or movements.

In certain aspects, the fat-soluble active of the extended release component, such as the extended release beads (including extended release beads, delayed release beads, or beads providing both extended and delayed release) and/or the extended release lipid multiparticulate, is optionally present at a weight percent of the extended release component of about 5% to about 95%, or any value or range therebetween. For example, the fat-soluble active(s) of the extended release component is optionally present at a weight percent of about 1% to about 15%, about 1% to about 25%, about 10% to about 35%, about 25% to about 45%, about 25% to about 55%, or any value or range therebetween. Optionally, the fat-soluble active(s) of the extended release component is present at 1% to 55% by weight, including any value or range therebetween. Optionally, the fat-soluble active(s) is present at 0.1% to 20% by weight, including any value or range therebetween. Optionally, the fat-soluble active(s) are present at 50-60%, with the rest of the extended release component comprising a combination of fats and waxes.

In certain aspects, the optional one or more water soluble vitamins and minerals, the one or more oil soluble vitamins and minerals, the performance enhancing component, and combination thereof of the extended release component such as the extended release beads (including extended release beads, delayed release beads, or beads providing both extended and delayed release) and/or the extended release lipid multiparticulate is optionally present at a weight percent of the composition of about 5% to about 95%, or any value or range therebetween. For example, the one or more water soluble vitamins and minerals, the one or more oil soluble vitamins and minerals, the performance enhancing component, and combination thereof of the extended release component is optionally present at a weight percent of about 1% to about 15%, about 1% to about 25%, about 10% to about 35%, about 25% to about 45%, about 25% to about 55%, or any value or range therebetween. Optionally, the one or more water soluble vitamins and minerals, the one or more oil soluble vitamins and minerals, the performance enhancing component, and combination thereof of the extended release component is present at 1% to 55% by weight, including any value or range therebetween. Optionally, the one or more water soluble vitamins and minerals, the one or more oil soluble vitamins and minerals, the performance enhancing component, and combination thereof is present at 0.1% to 20% by weight, including any value or range therebetween.

In some aspects, the extended release component, such as the extended release beads (including extended release beads, delayed release beads, or beads providing both extended and delayed release) and/or the extended release lipid multiparticulate, can comprise a barrier coating. A barrier coat comprises a water-permeable, water-insoluble, non-ionic polymer or co-polymer that confers either extended release or delayed release properties to the extended release component. In one aspect, the barrier coat can be applied, e.g., as an aqueous suspension or dispersion, over the extended release component, such as the extended release beads (including extended release beads, delayed release beads, or beads providing both extended and delayed release) and/or the extended release lipid multiparticulate, and forms a separate layer thereon. In some aspects, the barrier coat is directly over the extended release component, such as the extended release beads (including extended release beads, delayed release beads, or beads providing both extended and delayed release) and/or the extended release lipid multiparticulate, i.e., there are no intervening layers between the barrier coat and the beads or lipid multiparticulate. Depending upon the polymeric material selected, the barrier coat polymer or co-polymer may be cured (e.g., poly-vinyl acetate or ethylcellulose-based coatings). In certain aspects, a poly-vinyl acetate based coating may further include a plasticizer. In certain aspects, the barrier coating can comprise poly-vinyl acetate-based coatings, ethylcellulose-based coatings (e.g. SURELEASE™), hydrophobic shellac coatings, or enteric coatings, as are known in the art.

In certain aspects, a barrier coating can comprise an enteric coating. Enteric coatings can be used to manufacture delayed release beads or delayed release lipid multiparticulates. Suitable enteric coating materials include one or more polymers, for example but not limited to, methacrylic acid copolymers, cellulose acetate butyrate, cellulose acetate trimellitate, carboxymethylethylcellulose, shellac, Eudragit L, Eudragit S (Rohm Pharma), hydroxypropyl methylcellulose acetate succinate, hydroxypropyl methylcellulose phthalate, polyvinyl acetate phthalate, cellulose acetate phthalate.

Other barrier coatings can be utilized, e.g., the barrier coatings described in U.S. Pa. No. 6,066,334 and U.S. Pa. No. 6,046,277, U.S. Pa. No. 6,046,277, U.S. Pa. No. 6,001,392, US2007/0215511, US2005/232986, US2005/232987 US2005/232993, US2005/266032, and US2003/009971. Barrier coatings may be applied by a number of traditional methods including, but no limited to, conventional coating procedures or fluid bed spraying.

The total amount of the barrier coating present may vary within a wide range, optionally from about 0.1% by weight to about 20% by weight, including about 1% to about 15% by weight, about 5% to about 15% by weight, about 2% to about 10% by weight, and about 2% by weight to about 7.5% by weight of the total composition, including about 1%, 2%, 5%, 7.5%, 10%, 15%, and 20% by weight and ranges encompassing and bordered by such amounts. The amount of the barrier coating component(s) present may depend, at least in part, upon the amount and identity of each of the other components present (e.g. the amounts and physical characteristics of the extended release component, such as the extended release beads (including extended release beads, delayed release beads, or beads providing both extended and delayed release) and/or the extended release lipid multiparticulate, and any optional swellable polymer(s) and additives, and the identity and properties of the particular barrier coating component(s), with the object being to achieve a formulation which exhibits extended release or delayed release.

In some aspects, the extended release beads, including extended release beads, delayed release beads, or beads providing both extended and delayed release, may include one or more swellable polymers that act to modify, prolong, and/or slow the release over time of the at the extended release beads, including extended release beads, delayed release beads, or beads providing both extended and delayed release from the beads. A “swellable polymer” is a polymer that will swell in the presence of a dispersion medium, such as a fluid, optionally an aqueous fluid, optionally a digestive fluid of a mammal. Thus, swellable polymers are capable of absorbing water and physically swelling as a result, with the extent to which a polymer can swell being determined by the molecular weight or degree of crosslinking (for crosslinked polymers). The one or more swellable polymer is capable of swelling dimensionally unrestrained in upon contact with a dispersion medium, such as an aqueous medium. Suitable water-swellable polymers include those polymers that swell in a dimensionally unrestrained manner upon contact with water. Such polymers may also gradually erode over time. Examples of such polymers include polyalkylene oxides, such as polyethylene glycols, particularly high molecular weight polyethylene glycols; cellulose polymers and their derivatives including, but not limited to, methylcellulose, ethylcellulose (e.g. SURELEASE™, available from Colorcon as an aqueous ethyl cellulose dispersion containing water (70.6% w/w), ethylcellulose (18.8% w/w), ammonium hydroxide (4.4% w/w), a medium chain triglyceride (4.0% w/w), and oleic acid (2.2% w/w)), hydroxyalkyl celluloses, hydroxymethyl cellulose, hydroxyethyl cellulose, hydroxypropyl cellulose, hydroxypropyl methylcellulose (available from Dow Chemical Company), carboxymethylcellulose, microcrystalline cellulose (available from FMC); polysaccharides and their derivatives; chitosan; poly(vinyl alcohol); xanthan gum; maleic anhydride copolymers; poly(vinyl pyrrolidone); starch and starch-based polymers; maltodextrins;

poly(2-ethyl-2-oxazoline); poly(ethyleneimine); polyurethane; hydrogels; crosslinked polyacrylic acids (e.g., polymers of acrylic acid cross-linked with polyalkenyl ethers or divinyl glycol such as Carbomer 974P NF Carbopol® or other available Carbopol® polymers, available from Lubrizol Advanced Materials, Inc.); poly(ethylene oxide); and combinations or blends of any of the foregoing. In certain aspects, the one or more swellable polymers may increase to a size sufficient to be retained in the stomach for an extended period of time.

When the optional one or more swellable polymers is present in the extended release beads, including extended release beads, delayed release beads, or beads providing both extended and delayed release, the total amount present may vary within a wide range, preferably from about 0.1% by weight to about 50% by weight, including about 2% to about 40% by weight, about 10% to about 40% by weight, and about 2% by weight to about 20% by weight of the total composition, including about 5%, 10%, 15%, 20%, 30%, 40%, and 50% by weight, including any value or range therebetween. The amount of the one or more swellable polymer components present may depend, at least in part, upon the amount and identity of each of the other components present (the amounts and physical characteristics of the fat-soluble active(s), the one or more water soluble vitamins and/or minerals of the extended or delayed release beads, and any barrier coatings and additives, as well as the identity and properties of the particular polymer(s), with the object being to achieve a bead formulation which exhibits extended release and/or delayed release.

In some aspects, the extended release beads, including extended release beads, delayed release beads, or beads providing both extended and delayed release, may be milled, pressed or otherwise produced to achieve a desired average particle size or range defined as the particle size determined by the area-based measurement relative to an ideal sphere with the same surface area as the particle and typically measured using techniques such as optical granulometry. The average particle size of the extended release beads, including extended release beads, delayed release beads, or beads providing both extended and delayed release, can range from about 150 μm to about 2000 μm, or any value or range therebetween. In other aspects, the beads can be milled or passed through a sieve to provide an average particle size ranging from about 400 μm to about 1700 μm, or any value or range therebetween. In other aspects, the beads can be milled or passed through a sieve to provide an average particle size ranging from about 800 μm to about 1250 μm, or any value or range therebetween. These bead sizes may be determined using sieve analysis through a sieve shaker having USP standard wire mesh sieves conforming to ASTM specifications (e.g. 16, 20, 30, 40, 60, or 80 mesh screen, optionally a scree of 10 to 80 mesh). In some aspects, the extended release lipid multiparticulates may have an average particle size between 0.5 μm to about 1500 μm, or any value or range therebetween. In other aspects, the extended release lipid multiparticulates may have an average particle size between 10 μm to about 5000 μm, or any value or range therebetween.

In certain aspects the extended release component, such as the extended release beads (including extended release beads, delayed release beads, or beads providing both extended and delayed release) and/or the extended release lipid multiparticulate, optionally include one or more additives including but not limited to, e.g., one or more of a diluent, binder, lubricant, disintegrant, stabilizer, surfactant, glidant, sweetener, a preservative, sodium citrate; silica; flavoring agents, coloring agents, preservatives, or other components (e.g., water, such as but not limited to potable water; and pigments, such as but not limited to titanium dioxide). The choice of which such materials to us, if any, and the amounts to be utilized are considered to be within the abilities of one of skilled in the art, in view of the disclosure herein.

Exemplary diluents may include, but are not limited to calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol

Exemplary binders may include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Examples of polyvinylpyrrolidone include povidone, copovidone and crospovidone.

Exemplary lubricants may include, but are not limited to magnesium stearate, calcium stearate, stearic acid, and hydrogenated vegetable oil (e.g. comprising hydrogenated and refined triglycerides of stearic and palmitic acids).

Exemplary disintegrants may include, but are not limited to starches, sodium starch glycolate, croscarmellose sodium, clays, celluloses, algins, gums, or crosslinked polymers (e.g., crosslinked polyvinyl pyrrolidone), alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, pregelatinized starch, low-substituted hydroxypropyl cellulose.

Fillers include, for example, materials such as kaolin, powdered cellulose, and microcrystalline cellulose, as well as soluble materials such as mannitol, urea, sucrose, lactose, lactose monohydrate, dextrose, sodium chloride, and sorbitol.

Exemplary sweeteners may include those sweeteners well known in the art, including both natural and artificial sweeteners. Thus, exemplary sweeteners may include water-soluble sweetening agents such as monosaccharides, disaccharides, and polysaccharides such as xylose, ribose, glucose, mannose, galactose, fructose, high fructose corn syrup, dextrose, sucrose, sugar, maltose, partially hydrolyzed starch, or corn syrup solids and sugar alcohols such as sorbitol, xylitol, mannitol and mixtures thereof. Additional exemplary sweeteners include optionally sugar or sugar substitute (e.g. sucralose (1,6-Dichloro-1,6-dideoxy-β-D-fructofuranosyl-4-chloro-4-deoxy-α-D-galactopyranoside), aspartame, and the like.

Exemplary preservatives may include sodium benzoate, benzoic acid, potassium sorbate, salts of edetate (also known as salts of ethylenediaminetetraacetic acid, or EDTA, such as disodium EDTA), parabens (e.g., methyl, ethyl, propyl or butyl-hydroxybenzoates, etc.), and sorbic acid. Other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3 -Diaminopropanetetraacetic acid (1,3 -PDTA); 2,2-ethylenedioxybis [ethyliminodi(acetic acid)] (EGTA); 1,10-bis(2-pyridylmethyl)-1,4,7,10-tetraazadecane (BPTETA); ethylenediamine (EDAMINE); Trans-1,2-diaminocyclohexane-N,N,N′,N′-tetraacetic acid (CDTA); ethylenediamine-N,N′-diacetate (EDDA); phenazine methosulphate (PMS); 2,6-Dichloro-indophenol (DCPIP); Bis(carboxymethyl)diaza-18-crown-6 (CROWN); porphine; chlorophyll; dimercaprol (2,3-Dimercapto-l-propanol); citric acid; tartaric acid; fumaric acid; malic acid; and salts thereof can be utilized as preservatives. Each preservative must be evaluated in each formulation to assure the compatibility and efficacy of the preservative. Methods for evaluating the efficacy of preservatives in compositions and formulations are known and routine to those skilled in the art.

Exemplary flavoring agents may include both natural and artificial flavors, mints such as peppermint, menthol, artificial vanilla, chocolate, cinnamon, various fruit flavors, both individual and mixed, essential oils (i.e. thymol, eucalyptol, menthol and methyl salicylate) and the like.

In some aspects, surfactants, super disintegrants, or combinations thereof can be included in the extended release lipid multiparticulates. The use of surfactants, super disintegrants, or combinations thereof can promote the breakup of the lipid mutliparticulate in an aqueous environment, thereby increasing the available surface area and promoting release of the fat-soluble active, as well as the optional water-soluble vitamins and minerals and performance enhancing component.

Exemplary surfactants useful for this purpose include sorbitan esters (Spans), ethoxylated sorbitan esters (Tweens), poloxamers, and lecithins. Exemplary sorbitan esters include sorbitan monolaurate (available commercially as Span 20 from Croda), sorbitan monopalmitate (available commercially as Span 40 from Croda), sorbitan monostearate (available commercially as Span 60 from Croda), sorbitan monooleate (available commercially as Span 80 from Croda), sorbitan sesquioleate (available commercially as Span 83 from Croda), sorbian trioleate (available commercially as Span 85 from Croda), sorbitan isostearate (available commercially as Span 120 from Croda), polyoxylglycerides (e.g., lauroyl polyoxyl-32 glycerides, available commercially as gelucire® 44/14 from Gattefosse) and combinations thereof. Exemplary ethoxylated sorbitan esters (Tweens), include polyethylene glycol (PEG)-20 sorbitan monolaurate (available commercially as Tween 20 from Croda), PEG-4 sorbitan monolaurate (available commercially as Tween 21 from Croda), PEG-20 sorbitan monopalmitate (available commercially as Tween 40 from Croda), PEG-20 sorbitan monostearate (available commercially as Tween 60 from Croda), PEG-4 sorbitan monostearate (available commercially as Tween 61 from Croda), PEG-20 sorbitan tristearate (available commercially as Tween 65 from Croda), and PEG-20 sorbitan monooleate (available commercially as Tween 80 from Croda). Exemplary poloxmers include poloxamers available under trade name Synperonic™ (available commercially from Croda), poloxamers available under the trade names Pluronics™ and Kolliphor™ (both available commercially by BASF). Exemplary super-disintegrants include modified starches such as sodium carboxymethyl starch (sodium starch glycolate), cross-linked polyvinylpyrrolidone such as crospovidone, soy polysaccharide, cross-linked alginic acid, gellan gum, xanthan gum, calcium silicate, and ion exchange resins such as Indion 414.

In some aspects, an extended release fat-soluble active composition that includes an extended release bead multiparticulate comprising a fat-soluble active can further comprise an immediate release oil phase. The immediate release oil phase comprises one or more edible oils and optionally one or more oil soluble vitamins and/or minerals, a performance enhancing component, such as a performance enhancing supplement, and various combinations thereof. Similarly, in some aspects, an extended release fat-soluble active composition that includes an extended release lipid multiparticulate comprising a fat-soluble active can further comprise an immediate release aqueous liquid phase. The immediate release aqueous liquid phase comprises one or more aqueous liquids that will not dissolve the lipid multiparticulates, and optionally one or more vitamins and/or minerals, performance enhancing component, such as a performance enhancing supplement, and various combinations thereof. In certain aspects, the aqueous liquid phase can comprise glycerol, sorbitol, and/or other sugar alcohols that will not dissolve the lipid multiparticulates, e.g., optionally erythritol, threitol, arabitol, xylitol, ribitol, mannitol, galactitol, fucitol, iditol, inositol, volemitol, isomalt, maltitol, lactitol, maltotriitol, maltotetraitol, polyglycitol, and combinations thereof.

The term “immediate release” is the release of the oil phase or aqueous liquid phase (including the one or more vitamins and/or minerals, the performance enhancing component, and combinations thereof), from the compositions where the rate of release is not retarded by means of a controlled release matrix, controlled release coating, or other such means. The immediate release performance enhancing supplement is designed such that, upon ingestion, maximum exposure of said one more vitamins and/or minerals, the performance enhancing component, and combinations thereof, from the composition to body tissues occurs in the minimum period of time. As described herein, an “immediate release” component optionally releases at least one more of the vitamins and/or minerals, the performance enhancing component, and combinations thereof in less than about 1 hr, in about 45 minutes, in about 30 minutes, in about 20 minutes, in about 10 minutes, in about 5 minutes, in about 3 minutes, in about 2 minutes, or as soon as about 1 minute. In some aspects, the oil phase or aqueous liquid phase (including the one more vitamins and/or minerals, the performance enhancing component, and combinations thereof), are suitable to enhance nutrition and/or exercise performance.

In certain aspects, the oil phase can comprise edible oils or components thereof, such as fatty acids and medium chain triglycerides. In some aspects, an edible oil is a fish oil, or optionally a bioactive component thereof. As used herein, “fish oils” are oils that are obtained either directly or indirectly from one or more aquatic life forms. Fish oil can be derived from fresh or salt water fish or shellfish. In certain aspects, fish oils are obtained from oily fish. Fish oils are high in one or more of omega-3 fatty acids, such as docosahexaneoic acid, eicosapentaenoic acid docosapentaenoic acid, eicosatetraenoic acid, moroctic acid and heneicosapentenoic acid relative to non-fish oils. Omega-3 fatty acids are beneficial for prevention of cardiovascular pathology, for reversal of atherosclerosis, for inhibition of tumor formation, and for regulation of cholesterol. In certain aspects, the one or more fish oils of the oil phase of the composition comprises docosahexaneoic acid and eicosapentaenoic acid.

It is appreciated that some aspects may include more than one edible oil, optionally 2, 3, 4, 5, 6, or more edible oils or bioactive components thereof. Illustrative additional or substitutable edible oils include, but are not limited to vegetable oils, such as, evening primrose oil, black currant seed oil, borage oil, borage seed oil, safflower oil, safflower seed oil, sunflower oil, sunflower seed oil, sesame seed oil, peanut oil, walnut oil, almond oil, olive oil, olive seed oil, avocado oil, avocado seed oil, pumpkin seed oil, corn oil, cod liver oil, soy oil, soybean oil, coconut oil, palm oil, palm kernel oil, rapeseed oil, flaxseed (linseed) oil, cotton seed oil, tung oil, palmolein oil, mustard seed oil, oiticica oil and castor oil, arachidonic acid, leichitin, and conjugated linoleic acids combinations thereof. Edible oils are commercially available from sources known by those of skill in the art. In certain aspects, the oil phase can be in a liquid or paste-like state at 25° C.

In certain aspects, the one or more edible oils of the oil phase of the extended release fat-soluble active composition can range from about 0.5 to about 90% by weight. In other aspects, the one or more edible oils of the oil phase of the composition can range from about 5% to about 50% by weight, including any value or range therebetween.

The one more oil soluble vitamins and/or minerals of the oil phase can comprise at least one of Vitamin A (optionally as Retinyl Palmitate), Vitamin D (optionally as

Cholecalciferol), Vitamin E (optionally as D-Alpha Tocopherol), Vitamin K (optionally as Phytonadione), Vitamin F and various mixtures or other combinations thereof. Oil soluble vitamins and/or minerals are commercially available from sources known by those of skill in the art.

Vitamins and/or minerals in the aqueous liquid phase can comprise can comprise at least one of Vitamin B1 (optionally in the form of Thiaine Mononitrate), Vitamin B2 (optionally in the form of Riboflavin), Vitamin B3 (optionally in the form of Niacin), Vitamin B5 (optionally in the form of Pantothenic Acid), Vitamin B6 (optionally in the form of Pyridoxine HCL), Folic Acid, Vitamin B12 (optionally in the form of Methylcobalamin), Vitamin C (optionally in the form of Ascorbic Acid), Biotin, Calcium, Iron (optionally in the form of Ferrous Sulfate Monohydate), Phosphorus, Sulfer, Zinc (optionally in the form of Zinc Oxide), Copper (optionally in the form o Cupric Oxide), Iodine (Optionally in the form of Potassium Iodine), Manganese (optionally in the form of manganese gluconate), Chromium (optionally in the form of Chromium Chelate), Molybdenum (optionally in the form of Molybdenum Chelate), Selenium (optionally in the form of Sodium Selenate), Choline, Fluoride, Chloride, Potassium, Sodium, and various mixtures or other combinations thereof. These vitamins and/or minerals are commercially available from sources known by those of skill in the art.

The one more vitamins and/or minerals in the oil phase or aqueous liquid phase can range from about 0.1 to about 99% by weight based on the total weight of the oil phase or aqueous liquid phase, including any value and range therebetween. In other aspects, one more vitamins and/or minerals in the oil phase or aqueous liquid phase can range from about 0.5 to about 40% by weight based on the total weight of the oil phase or aqueous liquid phase, including any value or range therebetween.

The immediate release performance enhancing supplement of the oil phase or aqueous liquid phase of the extended release fat-soluble active composition can comprise, illustratively, one or more a: vitamin (e.g. vitamin B12 (optionally in the form of methycobalamin available from Anmar), vitamin B3 (optionally in the form of nicinaminde/nicotinamide available from DSM) among others); beta-alanine or derivative thereof (optionally CARNOSYN available from Natural Alternatives Intl.), mineral; protein; amino acid (e.g. arginine (optionally in the form of arginine silicate inositol available as Nitrosigine from Nutrition 21, or agmatine sulfate available from Parchem) glutamine (available from Kyowa Hakko), creatine (optionally in the form of creatine HCL available from Pharmline), carnitine, glycine, trimethyl glycine, tyrosine, leucine (available from Glanbia or Danisco), isoleucine (available from Glanbia), valine (available from Glanbia), citrulline (optionally in the form of citrulline malate available from Creative Compounds), among others or derivatives thereof optionally N-acetyl L-tyrosine (available from Cepham); branched-chain amino acids (optionally in the form of Pepform 2:1:1 BCAA containing a 2:1:1 ratio of leucine, isoleucine and valine, available from Glanbia); astragalus membranaceus and panax notoginseng carbohydrate (optionally in the form of Astragin available from N Liv Science); fatty acid (optionally essential fatty acid); stimulant illustratively caffeine (optionally 1,3,7-trimethylxanthine available from Mitsubishi), ephedrine, or forskholin; pyruvate; a citric acid cycle intermediate; betaine (optionally in the form of betaine anhydrous available from Danisco), norvaline (optionally in the form of L-novaline, available from Cepham), one or more plant components such as an essential oil or plant extract (illustratively citrus aurantium, grape seed extract, or piper nigrum (available from Indena)), black pepper extract (optionally BioPerine® or more specifically Black Pepper PE 95% BioPerine® from Sabinsa Corporation), ashwagandha extract (optionally KSM66 from Ixoreal)), or a derivative of any of the foregoing.

In certain aspects, the immediate release performance enhancing supplement of the oil phase or aqueous liquid phase is an uncoated performance enhancing supplement. Thus, the immediate release performance enhancing supplement can function to maintain vasodilation during and after a workout, stimulate muscle synthesis and repair over an extending period of time, and/or prevent athletic fatigue, such as sustaining energy levels and avoiding a subsequent energy level crash, when consumed by a subject.

The immediate release performance enhancing supplement of the oil phase or aqueous liquid phase of the extended release fat-soluble active composition is present to provide an in vivo concentration effective to function to improve athletic performance, such as maintaining vasodilation during and after a workout and stimulating muscle synthesis and repair over an extending period of time, and/or preventing athletic fatigue, such as sustaining energy levels and avoiding a subsequent energy level crash. In certain aspects, the immediate release performance enhancing supplement of the composition is optionally present at a weight percent of the composition of about 5% to about 95%, or any value or range therebetween. For example, the immediate release performance enhancing supplement of the composition is optionally present at a weight percent of about 5% to about 15%, about 15% to about 25%, about 25% to about 35%, about 35% to about 45%, about 45% to about 55%, about 55% to about 65%, about 65% to about 75%, about 75% to about 85%, about 85% to about 95%, or any value or range therebetween.

In certain aspects, the oil phase or aqueous liquid phase of the composition may further comprise one or more known additives/excipients such as preservative, flavorants, and colorants. In certain aspects, such additives/excipients, the amount thereof to be added optionally ranges from about 0.01 to about 5% by weight based on the total amount of the oil phase of the composition, including any value or range therebetween.

Exemplary preservatives that may be included in the oil phase or aqueous liquid phase of the composition include sodium benzoate, benzoic acid, potassium sorbate, salts of edetate (also known as salts of ethylenediaminetetraacetic acid, or EDTA, such as disodium EDTA), parabens (e.g., methyl, ethyl, propyl or butyl-hydroxybenzoates, etc.), and sorbic acid. Other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3 -PDTA); 2,2-ethylenedioxybis [ethyliminodi(acetic acid)] (EGTA); 1,10-bis(2-pyridylmethyl)-1,4,7,10-tetraazadecane (BPTETA); ethylenediamine (EDAMINE); Trans-1,2-diaminocyclohexane-N,N,N′,N ′-tetraacetic acid (CDTA); ethylenediamine-N,N′-diacetate (EDDA); phenazine methosulphate (PMS); 2,6-Dichloro-indophenol (DCPIP); Bis(carboxymethyl)diaza-18-crown-6 (CROWN); porphine; chlorophyll; dimercaprol (2,3-Dimercapto-1-propanol); citric acid; tartaric acid; fumaric acid; malic acid; and salts thereof can be utilized as preservatives. Each preservative must be evaluated in each formulation to assure the compatibility and efficacy of the preservative. Methods for evaluating the efficacy of preservatives in compositions and formulations are known and routine to those skilled in the art.

Exemplary flavorings that may be included in the oil phase or aqueous liquid phase of the composition include both natural and artificial flavors, and mints such as peppermint, menthol, artificial vanilla, chocolate, cinnamon, various fruit flavors, both individual and mixed, essential oils (i.e. thymol, eucalyptol, menthol and methyl salicylate) and the like.

The instantly-disclosed compositions can be administered by any desirable route. Optionally, the composition is administered orally. An administration time is optionally before, during, or following exercise. Optionally, the composition is administered orally prior to exercise or during exercise. In certain aspects, the composition is administered once a day. The oil phase or aqueous phase (including the one more vitamins and/or minerals, the performance enhancing component, and combinations thereof) and the extended release component (including extended release beads and/or extended release lipid multiparticulate) are subsequently mixed to form an oral dosage form. In particular aspects, the oil phase or aqueous phase (including the one more vitamins and/or minerals, the performance enhancing component, and combinations thereof) and the extended release component (including extended release beads and/or extended release lipid multiparticulate) are mixed and contained within a capsule, optionally forming a two-piece liquid capsule, or in a softgel.

As such, processes are provided enhancing and sustaining a supply of a fat-soluble active in a subject. Additionally, depending on the fat-soluble active(s) in the composition, processes are provided for enhancing nutrition, providing a protective effect or prevent various cancers, providing an analgesic effect, providing an anti-psychotic effect, relieving convulsions, relieving inflammation, relieving anxiety, relieving nausea, treating symptoms of multiple sclerosis, reducing the risk of a heart attack, reducing the risk of cardiovascular diseases, and decreasing the risk of developing amyotrophic lateral sclerosis, that include administering a the instantly-disclosed composition(s) as provided to a mammalian subject, optionally a human, wherein the administration is performed at a time suitable. Additionally, processes are provided for enhancing nutrition and/or athletic performance or preventing fatigue that include administering a the instantly-disclosed composition(s) as provided to a mammalian subject, optionally a human, wherein the administration is performed at a time suitable for enhancing athletic performance or preventing fatigue.

The foregoing description is illustrative of particular aspects of the invention, but is a limitation upon the practice thereof. In order that various aspects may be more readily understood, reference is made to the following examples which are intended to illustrate various aspects, but do not limit the scope thereof.

Various modifications of the present invention, in addition to those shown and described herein, will be apparent to those skilled in the art of the above description. Such modifications are also intended to fall within the scope of the appended claims.

It is appreciated that all reagents are obtainable from commercial sources known in the art unless otherwise specified.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference in its entirety.

The foregoing description is illustrative of particular aspects of the invention, but is not meant to be a limitation upon the practice thereof.

1. An extended release fat-soluble active composition comprising:
extended release beads comprising a fat-soluble active, the extended release beads configured to release greater than 80% of the fat-soluble active over a period of 4 hours or more.

2. The extended release fat-soluble active composition of claim 1, wherein the fat-soluble active comprises at least one of a fat-soluble vitamin, a carotenoid, a cannabinoid, a polyunsaturated fatty acid, or combinations thereof.

3. The extended release fat-soluble active composition of claim 2, wherein the cannabinoid is selected from the group consisting of a cannabinol, a cannabidiol, a tetrahydrocannabinol, and/or a combination thereof.

4. The extended release fat-soluble active composition of claim 1, wherein the extended release beads comprise an inert core and a nutrient layer coating the inert core, wherein the nutrient layer comprises the fat-soluble active.

5. The extended release fat-soluble active composition of claim 4, wherein the nutrient layer comprises one or more vitamins and/or minerals.

6. The extended release fat-soluble active composition of claim 4, wherein the nutrient layer comprises a performance enhancing supplement.

7. The extended release fat-soluble active composition of claim 4, wherein the extended release beads further comprise a seal film layer, wherein the seal film layer comprises a sealer and the seal film layer coats the nutrient layer.

8. The extended release fat-soluble active composition of claim 1, wherein the extended release beads further comprise a barrier coating layer.

9. The extended release fat-soluble active composition of claim 1, wherein the extended release beads further comprise one or more swellable polymers.

10-11. (canceled)

12. The extended release fat-soluble active composition of claim 1, wherein the particle size of extended release beads is from about 400 μm to about 1700 μm.

13. The extended release fat-soluble active composition of claim 1, wherein the particle size of extended release beads is from about 800 p.m to about 1250 μm.

14. The extended release fat-soluble active composition of claim 1, wherein the extended release beads further comprise a binder.

15. The extended release fat-soluble active composition of claim 14, wherein the nutritional layer of the extended release beads further comprises a binder.

16. The extended release fat-soluble active composition of claim 1, wherein the inert core of the extended release beads comprises at least one of celluloses, starches, saccharides, or mixtures thereof.

17. (canceled)

18. The extended release fat-soluble active composition f claim 1 further comprising a performance enhancing supplement, wherein the performance enhancing supplement comprises at least one of a vitamin, a mineral, protein, creatine, an amino acid, a carbohydrate, a fatty acid, a stimulant, pyruvate, black pepper extract, and a citric acid cycle intermediate.

19. The extended release fat-soluble active composition of claim 1, wherein the composition further comprises an immediate release oil phase, wherein the extended release beads are incorporated into the oil phase.

20. (canceled)

21. The extended release fat-soluble active composition of claim 19, wherein the immediate release oil phase comprises one or more edible oils.

22. The extended release fat-soluble active composition of claim 19, wherein the oil phase comprises one or more vitamins and/or minerals and/or a performance enhancing supplement.

23. An extended release fat-soluble active composition comprising:
extended release lipid multiparticulates comprising a fat-soluble active and a lipid matrix, the lipid matrix configured to release 80% or more of the fat-soluble active over a period of 4 hours or more, and optionally one or more vitamins and/or minerals and a performance enhancing supplement.

24-37. (canceled)

38. The extended release fat-soluble active composition of claim 1, wherein the composition is in the form of a liquid capsule.
